(19) United States
(12) Patent Application Publication
(10) Pub. No.: US 2013/0211205 A1
(43) Pub. Date: Aug. 15, 2013

(54) IMPLANTABLE MEDICAL DEVICE ORIENTATION CHANGE DETECTION

(71) Applicants: William Havel, West Lafayette, IN (US); Raja N. Ghanem, Edina, MN (US)

(72) Inventors: William Havel, West Lafayette, IN (US); Raja N. Ghanem, Edina, MN (US)

(73) Assignee: Medtronic, Inc.

(21) Appl. No.: 13/372,840

(22) Filed: Feb. 14, 2012

(51) Int. Cl. A61B 5/11; A61N 1/39; A61B 5/00

(52) U.S. Cl. 600/301; 607/7

(57) ABSTRACT

A medical device including a housing, multiple sensing elements positioned along the housing for use in sensing a physiological signal, and an accelerometer is configured to detect a change in device orientation relative to patient anatomy. The device measures a first accelerometer signal corresponding to a first orientation of the housing with respect to a patient position. The device detects a second orientation of the housing different than the first orientation in response to a comparison between the first accelerometer signal and a next accelerometer signal.

### FIELD OF THE DISCLOSURE

The disclosure relates generally to medical devices and, in particular, to an apparatus and method for detecting a change in orientation of an implanted medical device having housing-based sensors.

### BACKGROUND

Subcutaneous sensing of ECG signals is possible using implantable medical devices (IMDs) having sensing electrodes incorporated along the IMD housing or “CAN”. Placing sensing electrodes on a CAN surround allows collection and recording of ECG signals for clinical diagnostic purposes and for detecting a heart rhythm for use in controlling automatic device-delivered therapies, such as defibrillation shock pulses. During implantation of the IMD, it is expected that heart rhythm detection and diagnostic features will be programmed or optimized assuming a specific IMD orientation in the patient's body. For example, in a device with multiple CAN surround electrodes, a specific electrode pair would be used to emulate the Lead II ECG signal. Similarly, a detection method using surround electrodes might use an optimal measurement vector determined during implant providing a greatest signal-to-noise ratio or other signal quality measurement. It is possible, however, that after implantation, IMD rotation with respect to the patient's anatomy could occur, potentially resulting in changes in the directionality and/or signal quality of the ECG signals.

### BRIEF DESCRIPTION OF THE DRAWINGS

FIG. 1 depicts one example of a subcutaneous implantable cardioverter defibrillator (SubQ ICD) in which the presently disclosed techniques may be embodied.

FIG. 2 is a top and plan view of the SubQ ICD shown in FIG. 1.

FIG. 3 is a block diagram of electronic circuitry of the SubQ ICD of FIG. 1.

FIG. 4 is a flow chart of a method for detecting a change in orientation of a medical device housing and responding thereto.

### DETAILED DESCRIPTION

In the following description, references are made to illustrative embodiments. It is understood that other embodiments may be utilized without departing from the scope of the disclosure. As used herein, the term “module” refers to an application specific integrated circuit (ASIC), an electronic circuit, a processor (shared, dedicated, or group) and memory that execute one or more software or firmware programs, a combinational logic circuit, or other suitable components that provide the described functionality.

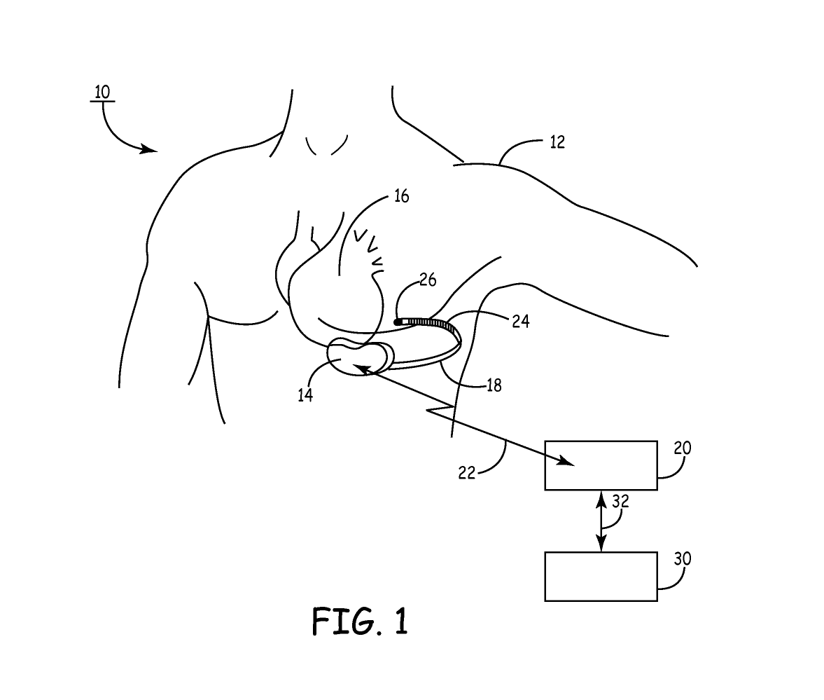

FIG. 1 depicts one example of an implantable medical device system 10 in which the presently disclosed techniques may be embodied. System 10 includes a subcutaneous implantable cardioverter defibrillator (SubQ ICD) 14 shown implanted subcutaneously in a patient 12, outside the ribcage and anterior to the cardiac notch. A subcutaneous lead 18 carrying a sensing electrode 26 and a high-voltage, cardioversion defibrillation coil electrode 24, is electrically coupled at its proximal end to SubQ ICD 14. The distal end of lead 18 is tunneled subcutaneously into a location adjacent to a portion of the latissimus dorsi muscle of patient 12. Specifically, lead 18 is tunneled subcutaneously from the median implant pocket of SubQ ICD 14 laterally and posterially to the patient's back to a location opposite the heart such that the heart 16 is generally disposed between the SubQ ICD 14 and distal electrode coil 24 and distal sensing electrode 26.

An external device 20 is shown in telemetric communication with SubQ ICD 14 by RF communication link 22. External device 20 may be a programmer, home monitor, hand-held or other device adapted to communicate with SubQ ICD 14. Communication link 22 may be any appropriate RF link, including Bluetooth, WiFi, MICS, or other communication protocol adapted for use with implantable medical devices.

External device 20 may be Internet enabled or coupled to a communication network 32 to allow communication between external device 20 and a networked device 30. Networked device 30 may be a Web-based centralized patient management database, a computer, a cell phone or other hand-held device. Networked device 30 communicates with external device 20 via communications network 32, which may be an Internet connection, a local area network, a wide area network, a land line or satellite based telephone network, or cable network. Networked device 30 may be used to remotely monitor and program SubQ ICD 14 via external device 20. Systems and methods for remotely communicating with an implantable medical device are generally disclosed in U.S. Pat. No. 5,752,976 to Duffin et al., U.S. Pat. No. 6,480,745 to Nelson et al., and U.S. Pat. No. 6,418,346 to Nelson et al., and U.S. Pat. No. 6,250,309 to Krichen et al., all of which patents are hereby incorporated herein by reference in their entirety.

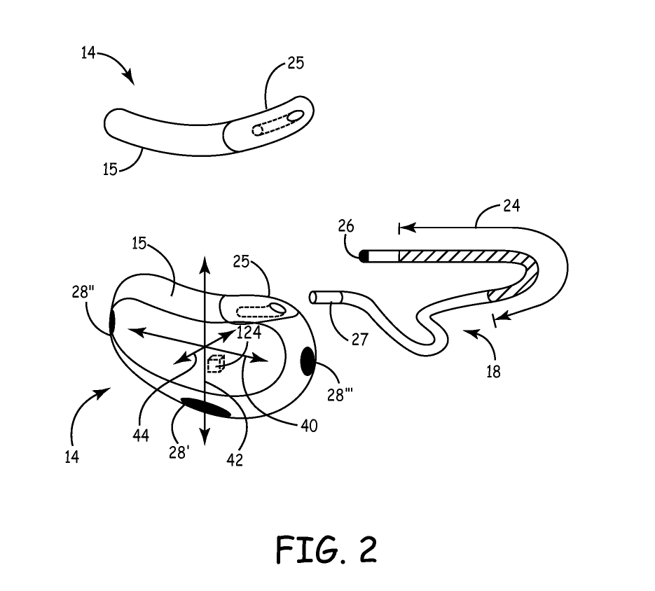

FIG. 2 is a top and plan view of SubQ ICD 14. SubQ ICD 14 includes a generally ovoid housing 15 having a substantially kidney-shaped profile. Connector block 25 is coupled to ICD housing 15 for receiving the connector assembly 27 of subcutaneous lead 18. SubQ ICD housing 15 may be constructed of stainless steel, titanium or ceramic. Electronics circuitry enclosed in housing 15 of SubQ ICD 14 may be incorporated on a polyamide flex circuit, printed circuit board (PCB) or ceramic substrate with integrated circuits packaged in leadless chip carriers and/or chip scale packaging (CSP). The plan view shows the generally ovoid construction of housing 15 that promotes ease of subcutaneous implant. This structure is ergonomically adapted to minimize patient discomfort during normal body movement and flexing of the thoracic musculature. The techniques described herein for detecting rotation of the implanted device, however, may be implemented in any implantable medical device housing shape and are not limited to the generally ovoid construction shown in FIG. 2.

Subcutaneous lead 18 includes distal coil electrode 24, distal sensing electrode 26, an insulated flexible lead body and a proximal connector assembly 27 adapted for connection to SubQ ICD 14 via SubQ ICD connector block 25. SubQ ICD 14 includes a housing-based subcutaneous electrode array (SEA) include electrodes 28′, 28″ and 28′″, collectively 28. SEA 28 includes multiple electrodes mounted along the housing 15. Three electrodes positioned in an orthogonal arrangement are included in SEA 28 in the embodiment shown in FIG. 2. Other embodiments of an IMD including housing-based electrodes may include any number of electrodes mounted on or incorporated along housing 15. It is recognized that any combination of lead-based and/or housing based electrodes may be used for sensing subcutaneous ECG signals. Multiple subcutaneous electrodes are provided to allow multiple subcutaneous ECG sensing vector configurations.

Electrode assemblies included in SEA 28 may be welded into place on the flattened periphery of the housing of SubQ ICD 14. The complete periphery of the SubQ ICD may be manufactured to have a slightly flattened perspective with rounded edges to accommodate the placement of SEA assemblies. In one embodiment, the SEA electrode assemblies are welded to SubQ ICD housing 15 (in a manner that preserves hermaticity of the housing 15) and are connected via wires (not shown in FIG. 2) to internal electronic circuitry (described herein below) inside housing 15. SEA electrode assemblies may be constructed of flat plates, or alternatively, spiral electrodes as described in U.S. Pat. No. 6,512,940 “Subcutaneous Spiral Electrode for Sensing Electrical Signals of the Heart” to Brabec, et al.

In other embodiments, the SEA 28 may be mounted in a non-conductive surround shroud, for example as generally described in U.S. Pat. No. 6,522,915 “Surround Shroud Connector and Electrode Housings for a Subcutaneous Electrode Array and Leadless ECGs” to Ceballos, et al. or in U.S. Pat. No. 6,622,046 “Subcutaneous Sensing Feedthrough/Electrode Assembly” to Fraley, et al., all of which patents are hereby incorporated herein by reference in their entireties.

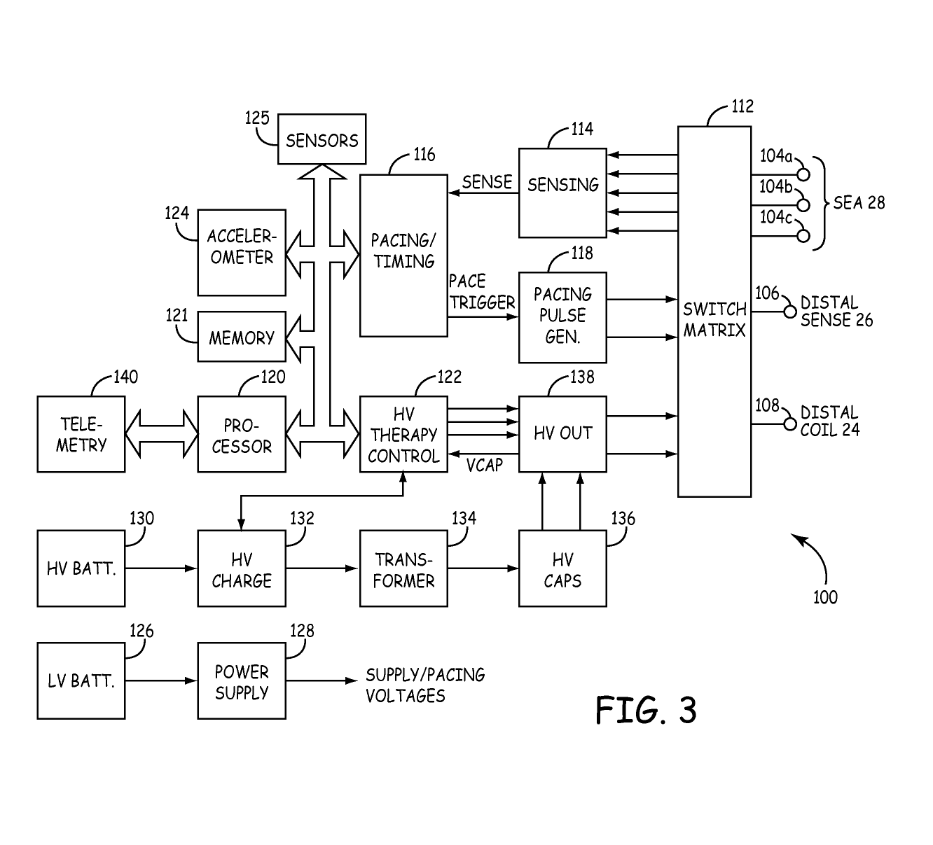

The electronic circuitry employed in SubQ ICD 14 detects a tachyarrhythmia from the sensed ECG signals and provides cardioversion/defibrillation shocks and may provide post-shock pacing as needed while the heart recovers. A block diagram of such circuitry adapted to function using subcutaneous sensing and cardioversion/defibrillation electrodes as described herein is shown in FIG. 3.

SubQ ICD 14 provides one illustrative embodiment of an IMD that includes electrodes disposed on or along a device housing. In other embodiments, one or more electrodes may be included along the housing of an IMD that is provided as a monitoring-only device that does not necessarily include therapy delivery capabilities such as cardiac pacing or cardioversion/defibrillation shock therapy. For example, an IMD including housing-based, i.e. “CAN” electrodes, for sensing electrical signals in a patient's body may include an ECG loop recorder, a hemodynamic monitor, a pacemaker, a drug delivery device, a neurostimulator, an electromyogram monitoring device or an electroencephalogram monitoring device. The techniques disclosed herein may be used with any IMD that includes one or more CAN electrodes used in recording electrical signals in the patient's body. If the IMD rotates with respect to an initial implant position, an electrode sensing vector utilizing at least one CAN electrode may change in directionality and/or signal strength or quality. As such, techniques described herein provide for detection of a change in orientation of the IMD housing relative to the patient's anatomy. In response to detecting a change in IMD position, an electrode sensing vector selection algorithm is performed to re-determine an optimal sensing vector using housing-based electrodes.

In the case of a single housing-based electrode, the single housing based electrode may be paired with a lead-based electrode extending away from the IMD. Multiple electrodes may be carried by one or more leads extending from the IMD. One of the multiple lead-based electrodes may be selected with the housing-based electrode for sensing electrical signals. If the IMD housing rotates with respect to the patient's anatomy, the initially selected sensing vector may no longer be optimal. A different lead-based electrode may be selected with the housing-based electrode in response to detecting rotation of the IMD according to the methods described herein.

Techniques described herein may be implemented in an implantable medical device that includes other types physiological sensors, in addition to or in place of housing-based electrodes. Multiple housing-based physiological sensors may be implemented along an IMD housing, directly in or on the IMD housing or along a surround shroud. One or more of the sensors may be selected as the optimal sensor(s) for monitoring a physiological signal. If rotation of the IMD is detected, the initially selected sensor(s) may no longer be optimal. A different sensor may be selected for monitoring the physiological signal in response to detecting IMD rotation. For example, multiple optical sensors, such as optical sensors used for monitoring tissue oxygen saturation, multiple pressure sensors, multiple acoustical sensors, or other type of sensors may be implemented along the housing for monitoring a physiological signal. In some embodiments, housing-based electrodes may be used for monitoring impedance signals. The techniques described herein may be implemented in conjunction with any IMD system incorporating housing-based sensors that provide any type of physiological signal that is influenced by position or orientation of the housing-based sensor. In other words, any housing-based sensor producing a signal that is altered due to a change in the position of the housing relative to the patient's anatomy may be implemented in conjunction with the techniques described herein.

SubQ ICD 14 includes a three-dimensional accelerometer 124 in one embodiment comprising three orthogonally configured accelerometers substantially aligned with the three-dimensional device axes 40, 42 and 44. For example, an x-axis solid state DC accelerometer, a y-axis solid state DC accelerometer and a z-axis solid state DC accelerometer may each be mounted along a hybrid circuit substrate within housing 15. The sensitive axes of DC accelerometers are orthogonally directed to one another and are aligned with the X, Y and Z device axes 40, 42 and 44. In relation to a standing patient, these X, Y and Z device axes 40, 42 and 44 may correlate to superior-inferior (S-I), lateral-medial (L-M) and anterior-posterior (A-P) body axes, respectively. The physician can implant and stabilize the SubQ ICD so that the X, Y and Z device axes 40, 42 and 44 are aligned as closely as possible to the corresponding S-I, A-P, and L-M body axes of the patient, though such alignment is not required for the purposes of detecting device rotation. Any type of accelerometer that can provide a DC output signal that changes with its orientation or position sensitive to the earth's gravity would be usable.

FIG. 3 is a block diagram of electronic circuitry 100 of SubQ ICD 14. Circuitry 100, which is located within housing 15, includes terminals 104A-104C, 106 and 108; switch module 112; sensing module 114; pacing timing and control circuit 116; pacing pulse generator 118; processor and control 120; memory 121, high voltage (HV) therapy control 122; accelerometer 124; low-voltage battery 126; power supply 128; high-voltage battery 130; high-voltage charging circuit 132; transformer 134; high-voltage capacitors 136; high-voltage output circuit 138; and telemetry circuit 140.

Electrodes included in SEA 28 are connected to terminals 104A-104C. Terminal 106 is connected to distal sense electrode 26 of subcutaneous lead 18. Distal sense electrode 26 may be used for sensing ECG signals and/or in delivery pacing pulses. SEA electrodes 28 and distal sense electrode 26 may be used as sensing electrodes to supply ECG input signals through switch module 112 via terminals 104A through 104C and 106 to sensing module 114. In some embodiments, as SEA 28 may be used as pacing electrodes to deliver pacing pulses from pacing pulse generator 118 through switch module 112. Terminal 108 is used to supply a high-voltage cardioversion or defibrillation shock from high-voltage output circuit 138 to coil electrode 24. In some embodiments, coil electrode 24 may be also used in sensing ECG signals.

Switch module 112 may include a switch array, switch matrix, multiplexer, or any other type of switching device suitable to selectively couple a signal to selected electrodes. Processor and control 120 and/or pacing timing and control circuit 116 may use the switch module 112 to select, e.g., via a data/address bus, which of the available electrodes of SEA 28 and electrodes 26 and 28 are used to deliver pacing pulses and coupled to pacing pulse generator 118 and which of SEA electrodes 28 and electrodes 26 and 28 are used for sensing ECG signals and coupled to sensing module 114.

Sensing module 114 and pacing timing and control 116 process the ECG signals. Signal processing may be performed on a transthoracic ECG signal from distal sense electrode 22 to an active CAN electrode, formed as a portion or all of IMD housing 15, or a single SEA electrode 28. Signal processing may additionally or alternatively be performed on a housing-based ECG signal defined by a selected pair of SEA electrodes 28, or a virtual vector based upon signals from all three SEA electrodes 28. Both the transthoracic ECG signal and the housing-based ECG signal are amplified and bandpass filtered by preamplifiers, sampled and digitized by analog-to-digital converters, and stored in temporary buffers.

Bradycardia is determined by pacing timing and control circuit 116 based upon R waves sensed by sensing circuit 114. An escape interval timer within pacing timing circuit 116 or HV therapy control 122 establishes an escape interval. Pace trigger signals are applied by pacing timing circuit 116 to pacing pulse generator 118 when the interval between successive R waves sensed is greater than the escape interval.

Detection of malignant tachyarrhythmia is determined by HV therapy control circuit 122 as a function of the intervals between R wave sense event signals from pacing timing circuit 116 and may employ ECG morphology analysis or other tachycardia or fibrillation detection algorithms. Processor 120, which may be embodied as a programmable microprocessor with associated memory 121 such as RAM and ROM storage, may store and execute algorithms for detecting arrhythmias and controlling device delivered therapies. Detection criteria used for tachycardia detection may be downloaded from external programmer 20 through telemetry circuit 140 and stored by memory 121.

Processor and control unit 120 may include any one or more of a microprocessor, a controller, a digital signal processor (DSP), an application specific integrated circuit (ASIC), a field-programmable gate array (FPGA), or equivalent discrete or integrated logic circuitry. In some examples, processor 120 may include multiple components, such as any combination of one or more microprocessors, one or more controllers, one or more DSPs, one or more ASICs, or one or more FPGAs, as well as other discrete or integrated logic circuitry. The functions attributed to processor and control 120, pacing timing and control 116 and HV therapy control 122 herein may be embodied as software, firmware, hardware or any combination thereof.

Memory 121 may include computer-readable instructions that, when executed by processor 120, cause SubQ ICD 14 to perform various functions attributed throughout this disclosure to ICD 14, processor 120, pacing timing and control 116, and HV therapy control 122. The computer-readable instructions may be encoded within memory 121. Memory 121 may comprise non-transitory computer-readable storage media including any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Memory 121 stores algorithms, intervals, counters, or other data used by processor and control 120 to control ICD functions. In particular, memory 121 is used to store accelerometer signal data for establishing an initial orientation of ICD 14 and for updating orientation data when a change in orientation is detected based an analysis of signals from accelerometer 124 by processor and control 120.

Processor and control 120 periodically or upon command measures an output signal and may derive a positional angle of each x-, y- and z-axis accelerometer to determine if a change in the accelerometer output or derived angle with respect to a previously stored signal data or derived angle is detected. A detected change will cause processor 120 to issue an electrode selection signal. The electrode selection signal is an indication that a positional change of the ICD has been detected and may have caused a change in a sensing electrode vector and/or a therapy delivery electrode vector. Electrode vector selection, ECG signal analysis, or capture threshold testing may be required to re-determine an optimal electrode vector for ECG sensing and/or therapy delivery.

The issued signal may be responded to by processor and control 120 itself by performing an automated ECG electrode sensing vector selection algorithm or performing a pacing capture threshold test or other diagnostic testing in accordance with the use of the housing-based electrodes or other type of housing-based sensors. Additionally or alternatively, the issued signal may be transmitted to programmer 20 via telemetry 140 to alert the patient and/or a clinician or other user that possible reprogramming of a sensing vector and/or therapy delivery parameters may be required.

Low-voltage battery 126 and power supply 128 supply power to circuitry 100. In addition, power supply 128 charges the pacing output capacitors within pacing pulse generator 118. High-voltage required for cardioversion and defibrillation shocks is provided by high-voltage battery 130, high-voltage charging circuit 132, transformer 134, and high-voltage capacitors 136. When a malignant tachycardia is detected, high-voltage capacitors 136 are charged to a preprogrammed voltage level by charging circuit 132 based upon control signals from control circuit 122.

Feedback signal Vcap from HV output circuit 138 allows HV control circuit 122 to determine when high-voltage capacitors 136 are charged. If the tachycardia persists, control signals from control 122 to high-voltage output signal 138 cause high-voltage capacitors 136 to be discharged through the body between distal coil electrode 24 and an active CAN electrode formed by housing 15.

Telemetry circuit 140 allows SubQ ICD 14 to be programmed by external programmer 20 through a bidirectional telemetry link. Uplink telemetry allows device status and other diagnostic/event data to be sent to external programmer 20 and reviewed by the patient's physician. Downlink telemetry allows external programmer 20, under physician control, to program ICD functions and set detection and therapy parameters for a specific patient.

ICD 14 may include additional sensors 125 such as a patient activity sensor, a real time clock, a temperature sensor, an optical sensor for measuring tissue oxygenation, or other types of physiological sensors. In some embodiments, an activity sensor signal and/or real time clock signal are used by processor and control 120 for verifying a patient position during a process for detecting a change in device orientation.

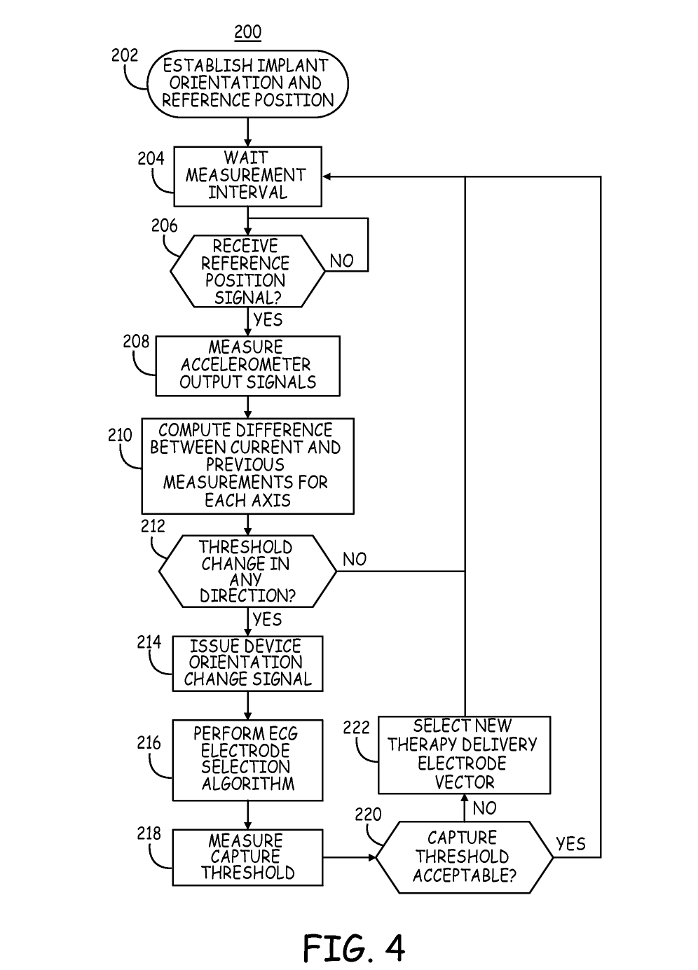

FIG. 4 is a flow chart 200 of a method for detecting a change in orientation of a medical device and responding thereto. Flow chart 200 is intended to illustrate the functional operation of the device, and should not be construed as reflective of a specific form of software or hardware necessary to practice the methods described. It is believed that the particular form of software, firmware and/or hardware will be determined primarily by the particular system architecture employed in the device and by the particular sensing and therapy delivery methodologies employed by the device. Providing software, firmware and/or hardware to accomplish the described functionality in the context of any modern medical device, given the disclosure herein, is within the abilities of one of skill in the art.

Methods described in conjunction with flow charts presented herein may be implemented in a non-transitory computer-readable medium that includes instructions for causing a programmable processor to carry out the methods described. A “non-transitory computer-readable medium” includes but is not limited to any volatile or non-volatile media, such as a RAM, ROM, CD-ROM, NVRAM, EEPROM, flash memory, or other computer-readable media, with the sole exception being a transitory, propagating signal. The instructions may be implemented as one or more software modules, which may be executed by themselves or in combination with other software.

The techniques described in conjunction with flow chart 200 are illustrated with reference to the SubQ ICD 14 incorporating housing-based ECG sensing electrodes, however, it is recognized that the disclosed techniques may be implemented in any implantable or externally worn device that includes electrodes or sensing elements used for sensing a physiological signal. Rotation of the device with may cause a change in the physiological signal being sensed, e.g. lower signal sensitivity, increased noise corruption of the sensed signal, or sensing of confounding physiological signal input due to rotation of the device. In the illustrative embodiments, the sensing elements carried along the ICD housing are electrodes used for sensing ECG signals. However, as discussed above, it is contemplated that in other embodiments, other sensing elements may be positioned along a device housing, such as optical sensing elements, temperature sensing elements, acoustical sensing elements, electrodes for measuring impedance, or other physiological sensors, that may change position relative to a targeted measurement volume of tissue or measurement vector due to device rotation. As such, the detection of device rotation may be used in causing re-selection of any type of housing-based sensors or combinations of sensors for sensing physiological signals.

It is further recognized that a housing-based sensing element may be used in conjunction with other housing-based sensing elements (e.g. a pair of housing-based electrodes) or a sensing element located away from the housing. For example, optical sensors may be arranged in a transmission configuration including a light source along the housing and a light detector located away from the housing configured to receive emitted light. In another example, a housing-based electrode may be paired with a lead-based electrode for measuring an electrical signal or an impedance signal. Rotation of the housing may require re-selection of the housing-based sensing element in such configurations to promote an optimal signal quality and/or targeted measurement vector or tissue volume.

At block 202, an initial device orientation and patient reference position are established. The initial device orientation may be established at the time of device implantation or may be established at a later follow-up. In order to detect device rotation with respect to the patient's anatomy, a reference body position of the patient is established. Repeated measurements of the device orientation are performed when the previously-established patient reference position is recognized or verified to allow comparable device orientation measurements to be obtained.

In one embodiment, the patient reference position is a prone, supine position. In other embodiments, the patient reference position may be a standing position or a sitting position. Depending on the type of device being used and its implant location, other body positions may be appropriate for the reference position. For example, if a device incorporating sensors along a device housing is implanted along a limb or extremity, a particular position of the limb or extremity is established. The patient may be instructed by a clinician to assume the reference position during an office visit or at regular intervals at home, for example, daily, weekly, monthly, bi-annually etc.

Initially, the patient is instructed to assume the reference position to enable measurement of the initial device orientation. A signal amplitude of each axis accelerometer is measured to obtain an initial device orientation. An angle of each axis of a multi-axis accelerometer may be derived from the measured signal amplitudes and stored in device memory. In one embodiment, if a three-dimensional accelerometer is implemented, an initial three-dimensional vector derived from the three-axis accelerometer is measured and stored in device memory. It is contemplated that in some embodiments, depending on the configuration of the device shape and location of the sensors, orientation and rotation around one device axis may critically affect sensed signals while orientation and rotation around another device axis may not be critical. As such, the dimensionality of the accelerometer used for measuring axial rotation of the device is determined based on the particular needs of the monitoring application.

Measurement of the initial orientation is generally performed by measuring the accelerometer output signal amplitude corresponding to each device axis along which an accelerometer axis is aligned. For example, if a 3D accelerometer includes orthogonal accelerometers aligned with an x-, y- and z-axis of the device, the accelerometer output signal may be used for computing the angle in the x-axis (pitch angle), the angle in the y-axis (yaw angle) and the angle in the z-axis (roll angle) from the respective amplitudes of the accelerometer signals when the patient has assumed the reference body posture or position.

In some embodiments, two or more reference body postures or positions are established at block 202 and assumed for obtaining respective sets of the accelerometer output signals. For example, two substantially orthogonal body positions, such as standing and supine positions or postures, may be assumed to obtain the accelerometer output signals in each axis for each posture. Processing and computation of initial orientation angles may be obtained in a method generally corresponding to the techniques described in U.S. Pat. No. 6,044,297 (Sheldon, et al.), hereby incorporated herein by reference in its entirety.

At block 204, the ICD processor and control waits for a measurement time interval. Computation of the device orientation angles may be performed at approximately regular intervals of time, for example daily, weekly, monthly, semi-annually etc. Upon expiration of the measurement time interval, the processor and control waits for a confirmation or verification signal that the patient has assumed the established reference position at block 206. The signal may be a user-entered signal transmitted to the ICD via telemetry using programmer 20. In other embodiments, the signal may be a tapping on the device by the patient, e.g. as generally disclosed in U.S. Pat. No. 5,836,975 (DeGroot), hereby incorporated herein by reference in its entirety.

In some embodiments, the verification signal that the patient is in the reference position may correspond to a real time clock signal and/or an activity sensor signal. For example, if the measurement time interval has expired, and an activity sensor signal indicates the patient is walking, the patient is assumed to be in a standing body posture. If an established reference position is an upright standing position, updated accelerometer output signals for each x-, y- and z-axis are measured at block 208 and stored in memory. The activity sensor signal output may be analyzed for verifying an established reference posture in conjunction with a designated range of daytime hours that the patient is expected to be awake.

In another example, if the device real time clock indicates a time of day as nighttime and/or an activity sensor signal output indicates a relatively long period, e.g. greater than one hour, of inactivity, a laying position or posture is verified. If an established reference position corresponds to a laying position, the accelerometer output signals are measured at block 208. The measurement time interval is optional in some embodiments wherein a real time clock signal and/or activity signal are used to trigger the recording of accelerometer output signals that are stored for comparison to previous output signals for detecting device rotation.

A difference between the updated accelerometer output signal measurements and previously stored accelerometer signal measurements is determined for each accelerometer axis (or for an overall 3D vector) at block 210. The differences are compared to a detection threshold at block 212 for detecting a significant rotation in device position with respect to the established reference patient position compared to the previous measurements. If no computed difference exceeds a detection threshold, the process returns to block 204 to wait for the next measurement time interval to expire.

If a threshold change is detected at block 212, a device orientation change signal is issued at block 214. In response to detecting the device orientation change, a sensor selection algorithm is performed at block 216. The selection algorithm may be performed automatically or in response to user input after the user has received the device orientation signal change via the ICD telemetry and an external programmer 20 or networked device 30.

In one embodiment, an ECG electrode selection algorithm may be performed for selecting a sensing vector having the greatest correlation to a desired surface ECG lead signal, e.g. a Lead II ECG signal. An ECG electrode selection algorithm may be performed to select a vector with minimal noise, highest signal-to-noise ratio, or other signal quality parameters. Examples of ECG electrode selection algorithms or signal quality metrics are generally described in U.S. Pat. No. 7,496,409 (Greenhut, et al.) and U.S. Pat. No. 7,904,153 (Greenhut, et al.), both of which are incorporated herein by reference in their entirety.

In some embodiments, after the ECG electrode selection algorithm has been performed, the process returns to block 204 to wait for the next measurement time interval. Alternatively, additional electrode evaluation may be performed. For example, if one or more electrodes located along the device housing are used for delivering an electrical stimulation therapy, which may be cardiac pacing or neurostimulation, the capture threshold may be measured at block 218 using the currently selected therapy delivery electrodes. A change in device orientation may cause a change in capture threshold and thus impact therapy delivery and effectiveness. If the capture threshold is still acceptable, as determined at block 220, the process returns to block 204. If the capture threshold is not acceptable, e.g. if the threshold has increased, a new therapy delivery electrode vector may be selected at block 222. The process then returns to block 204 to wait for the next measurement interval to expire.

Thus, a medical device and associated methods have been presented in the foregoing description with reference to specific embodiments. It is appreciated that various modifications to the referenced embodiments may be made without departing from the scope of the disclosure as set forth in the following claims.

1. A medical device system, comprising:
a housing enclosing a medical device;
a plurality of sensing elements positioned along the housing to sense a physiological signal;
an accelerometer positioned within the medical device to generate accelerometer signals corresponding to orientation of the device; and
a processor coupled to the accelerometer and configured to receive the accelerometer signals and determine a first accelerometer signal corresponding to a first orientation of the housing with respect to a patient position, determine a next accelerometer signal, compare the first accelerometer signal to the next accelerometer signal, detect whether there is a second orientation of the housing different than the first orientation in response to the comparison, and generate a device orientation change signal in response to detecting the second orientation.

2. The system of claim 1, wherein the processor is further configured to determine a change in sensing elements of the plurality of sensing elements for sensing a next physiological signal in response to the generated device orientation change signal.

3. The system of claim 1, further comprising a programming device configured to receive the device orientation change signal from the medical device, wherein the processor is configured to receive programming commands from the programming device for selecting which of the plurality of sensing elements are used for sensing the physiological signal.

4. The system of claim 1, wherein the accelerometer comprises a multi-axis accelerometer.

5. The system of claim 1, wherein the processor is configured to receive electrical signals from the plurality of sensing elements for use in detecting a patient condition.

6. The system of claim 5, wherein the processor is configured to select a first sensing vector comprising at least one of the plurality of sensing elements for sensing electrical signals and is further configured to automatically select a second sensing vector comprising at least one of the plurality of sensing elements for sensing electrical signals in response to detecting the second device orientation.

7. The system of claim 5, further comprising a therapy delivery module coupled to the plurality of sensing elements to deliver an electrical stimulation therapy, wherein the processor selects at least one of the plurality of sensing elements for use in delivering the electrical stimulation therapy in response to detecting the second device orientation.

8. The system of claim 1, further comprising means for detecting a predetermined patient position, wherein the processor measures the next accelerometer signal in response to detecting the predetermined patient position.

9. The system of claim 8, wherein the means for detecting a predetermined patient position comprises means for the processor to receive a user activated signal transmitted to the medical device.

10. The system of claim 8, wherein the means for detecting a predetermined patient position comprises one of an activity sensor and a real-time clock.

11. A method for determining orientation changes of a medical device, the method comprising:
determining a first accelerometer signal corresponding to a first orientation of the housing with respect to a patient position;
determining a next accelerometer signal;
comparing the first accelerometer signal to the next accelerometer signal;
detecting whether a second orientation of the device is different from the first orientation in response to the comparing; and
generating a device orientation change signal in response to detecting the second orientation.

12. The method of claim 11, further comprising determining a change in sensing elements of the plurality of sensing elements for sensing a next physiological signal in response to the generated device orientation change signal.

13. The method of claim 11, further comprising:
transmitting the device orientation change signal from the medical device to a programmer device; and
transmitting programming commands from the programming device to the processor for selecting which of the plurality of sensing elements are used in sensing the physiological signal.

14. The method of claim 11, further comprising generating multi-axis accelerometer signals, wherein the first and next accelerometer signals are determinined in response to the multi-axis accelerometer signals.

15. The method of claim 11, further comprising:
transmitting electrical signals from a plurality of sensing elements; and
detecting a patient condition in response to the transmitted electrical signals.

16. The method of claim 15, further comprising:
selecting at least one of a plurality of sensing elements in a first sensing vector for sensing electrical signals; and
automatically selecting a second sensing vector different from the first sensing vector and including at least one of the plurality of sensing elements for sensing electrical signals in response to detecting the second device orientation.

17. The method of claim 15, further comprising selecting at least one of the plurality of sensing elements for use in delivering an electrical stimulation therapy in response to detecting the second device orientation.

18. The method of claim 11, further comprising:
detecting a predetermined patient position; and
determining a next accelerometer signal in response to the detected predetermined patient position.

19. The method of claim 18, wherein detecting the predetermined patient position comprises receiving a user activated signal transmitted to the medical device.

20. The method of claim 18, wherein detecting the predetermined patient position comprises one of determining a patient activity level and determining a time of day.

21. A non-transitory, computer-readable medium storing instructions which cause a processor of a medical device to perform a method, the method comprising:
measuring a first accelerometer signal corresponding to a first orientation of the housing with respect to a patient position;
measuring a next accelerometer signal,
comparing the first accelerometer signal and the next accelerometer signal;
detecting a second orientation of the device different than the first orientation in response to the comparison; and
generating a device orientation change signal in response to detecting the second orientation.
